(19) United States
(12) Patent Application Publication
(10) Pub. No.: US 2023/0371867 A1
(43) Pub. Date: Nov. 23, 2023

(54) Blood Draw Device With Proximal Dual Port Diversion Chamber for Collection of a Blood Culture Sample From the PIV During Dwell

(71) Applicant: Becton, Dickinson and Company, Franklin Lakes, NJ (US)

(72) Inventor: Jonathan Karl Burkholz, Salt Lake City, UT (US)

(21) Appl. No.: 18/198,584

(22) Filed: May 17, 2023

Related U.S. Application Data
(60) Provisional application No. 63/343,772, filed on May 19, 2022.

(51) Int. Cl. A61B 5/15

(52) U.S. Cl. CPC A61B 5/150992; A61B 5/150213; A61M 25/0693

(57) ABSTRACT

A blood draw system including a blood draw device having an actuator and a blood draw tube operably coupled to the actuator, wherein the actuator is configured to selectively advance the blood draw tube through a catheter of a vascular access device, and an extension tube extending from the blood draw device, wherein the extension tube is in fluid communication with the blood draw tube of the blood draw device. The system also includes a dual port adapter coupled to a proximal end of the extension tube, wherein the dual port adapter includes a first port and a second port, and a vented flashback and diversion chamber coupled to one of the first port and the second port, wherein the vented flashback and diversion chamber is configured to receive and hold an initial volume of blood drawn through the extension tube by the blood draw device.

### CROSS-REFERENCE TO RELATED APPLICATION

The present application claims priority to U.S. Provisional Application Ser. No. 63/343,772 entitled “Blood Draw Device with Proximal Dual Port Diversion Chamber for Collection of a Blood Culture Sample from the PIV During Dwell”, filed May 19, 2022, the entire disclosure of which is hereby incorporated by reference in its entirety.

### BACKGROUND OF THE INVENTION

### Field of the Invention

The present disclosure generally relates to systems and methods for collecting a blood sample for blood culture testing from vascular access devices such as, e.g., peripheral intravenous catheters (PIVCs). More particularly, the systems described herein include blood draw devices with a proximal blood diversion chamber.

### Description of Related Art

When collecting a blood specimen from an indwelling vascular access devices, like a peripheral IV catheter (PIVC), a central venous catheter (CVC), or a peripherally inserted central catheter (PICC), the first 1-10 ml of blood is often wasted (i.e., disposed of) to avoid contamination from fluids in the dead space of the vascular access path of such devices. This extra step, while reducing contamination of the blood specimen, may be forgotten or improperly performed, thus compromising the blood specimen.

Blood cultures are often used as a tool to detect the presence of bacteria or fungi in a blood sample of a patient, to identify the type of bacteria or fungi present, and to direct the treatment of the patient. However, accidental contamination of the blood sample is a common problem, causing false positives and often resulting in a patient being prescribed unnecessary treatments such as, e.g., broad spectrum antibiotics. To address this concern, some healthcare providers clean the skin of the patient prior to a blood draw procedure. While this reduces the false positive rate, the rate is still significant (e.g., 3-5%) due to bacteria and/or fungi residing in, e.g., hair follicles. Therefore, some systems also divert a small volume of the initial blood drawn, with the initial (and potentially contaminated) volume being discarded. These systems, however, can be costly and time-consuming, and may only be used with an intravenous catheter at the immediate time of initial placement. Furthermore, these systems often rely on puncturing a patient's skin to collect the sample, which is uncomfortable for the patient.

Additionally, needle-free blood draw systems, such as PIVO™ from Becton, Dickinson and Company, are intended to be used in conjunction with indwelling intravenous catheters within the patient's vasculature to draw one or more blood samples directly from the vascular access device, avoiding the need for additional (and uncomfortable) venipunctures. However, insertion of the intravenous catheter into the patient's vasculature may introduce bacteria and/or fungi by contact with the patient's skin and dermal layers during the insertion process. Accordingly, the initial blood volume drawn into the needle-free blood draw system may contain the bacteria and/or fungi present solely due to catheter insertion, thereby increasing the risk of a false positive blood culture test. Furthermore, for indwelling catheters, microbes may be present within the vascular access device and may not necessarily indicate microbes within the blood stream. As the needle-free blood draw device advances through an indwelling catheter, microbes within the catheter device may enter the blood draw device, making it preferable to isolate the initial blood that enters the blood draw device into a diversion volume.

### SUMMARY OF THE INVENTION

In accordance with an aspect of the present disclosure, a blood draw system is disclosed. The blood draw system may include a blood draw device having a distal end portion and a proximal end portion, wherein the blood draw device includes an actuator and a blood draw tube operably coupled to the actuator, and wherein the actuator is configured to selectively advance the blood draw tube through a catheter of a vascular access device when the blood draw device is coupled to the vascular access device. The blood draw system may also include an extension tube extending from the proximal end portion of the blood draw device, wherein the extension tube is in fluid communication with the blood draw tube of the blood draw device, a dual port adapter coupled to a proximal end of the extension tube, wherein the dual port adapter includes a first port and a second port, and a vented flashback and diversion chamber coupled to one of the first port and the second port, wherein the vented flashback and diversion chamber is configured to receive and hold an initial volume of blood drawn through the extension tube by the blood draw device.

In some embodiments, the vented flashback and diversion chamber further includes a venting portion.

In some embodiments, the venting portion is configured to vent air when the blood draw device is coupled to the vascular access device and the blood draw tube of the blood draw device is advanced into a patient's vasculature to draw the initial volume of blood into the vented flashback and diversion chamber.

In some embodiments, the venting portion is configured to vent air automatically when the blood draw device is coupled to the vascular access device and the blood draw tube of the blood draw device is advanced into a patient's vasculature.

In some embodiments, the venting portion is manually vented when the blood draw device is coupled to the vascular access device and the blood draw tube of the blood draw device is advanced into a patient's vasculature.

In some embodiments, the venting portion is formed of at least one of a membrane, paper, porous material, film, or mechanical feature that allows air to pass therethrough but prevents fluid to pass therethrough when wetted.

In some embodiments, the system further includes a removable end cap positionable on the other one of the first port and the second port.

In some embodiments, the system further includes a luer lock access device coupled to the other one of the first port and the second port.

In some embodiments, the system further includes a secondary extension tube, wherein a distal end of the secondary extension tube is coupled to one of the first port and the second port and a proximal end of the secondary extension tube is coupled to a connector.

In some embodiments, the vented flashback and diversion chamber is coupled to the connector.

In some embodiments, the system further includes a fluid occlusion device positioned in-line with the secondary extension tube, wherein the fluid occlusion device is configured to selectively occlude the flow of blood from the vented flashback and diversion chamber.

In some embodiments, the blood draw device further includes an introducer body, and the actuator is configured to move linearly along the introducer body to advance and retract the blood draw tube from the distal end portion of the blood draw device.

In some embodiments, the vented flashback and diversion chamber and the second port have a combined internal volume of at least 0.15 mL.

According to another aspect of the present disclosure, a method of using a blood sample collection system is disclosed. The method may include providing the blood sample collection system, the system including a blood draw device having a distal end portion and a proximal end portion, wherein the blood draw device includes an actuator and a blood draw tube operably coupled to the actuator, an extension tube extending from the proximal end portion of the blood draw device, wherein the proximal extension tube is in fluid communication with the blood draw tube of the blood draw device, a dual port adapter coupled to a proximal end of the extension tube, wherein the dual port adapter includes a first port and a second port, and a vented flashback and diversion chamber coupled to one of the first port and the second port. The method may also include coupling the blood draw device to a vascular access device having an indwelling catheter, advancing the blood draw tube of the blood draw device through the vascular access device and beyond a distal tip of the indwelling catheter, and allowing the initial volume of blood to flow through the blood draw tube and the extension tube and into the vented flashback and diversion chamber of the blood sample collection system.

In some embodiments, the method may include venting the vented flashback and diversion chamber prior to allowing the initial volume of blood to flow therein.

In some embodiments, venting the vented flashback and diversion chamber may include manual venting.

In some embodiments, venting the vented flashback and diversion chamber may include automatic venting.

In some embodiments, the method may further include coupling a blood collection interface to the other one of the first port and the second port of the dual port adapter.

In some embodiments, the method may further include coupling a first blood collection container to the blood collection interface after the initial volume of blood is collected in the vented flashback and diversion chamber, and collecting a first blood sample within the first blood collection container.

In some embodiments, the method may further include removing the first blood collection container from the blood collection interface, coupling a second blood collection container to the blood collection interface, and collecting a second blood sample within the second blood collection container.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a plan view of a blood draw system with a proximal dual port blood diversion chamber in accordance with an aspect of the present disclosure;

FIG. 2 is a plan view of the blood draw system of FIG. 1 coupled to a vascular access device and luer lock access device in accordance with an aspect of the present disclosure;

FIG. 3 is a plan view of the blood draw system of FIG. 1 coupled to a vascular access device and luer lock access device in accordance with an aspect of the present disclosure;

FIG. 4 is a plan view of the blood draw system of FIG. 1 coupled to a vascular access device, luer lock access device, and blood culture collection tube in accordance with an aspect of the present disclosure;

FIG. 5 is a plan view of a blood draw system with a proximal dual port blood diversion chamber coupled to a vascular access device, luer lock access device, and blood culture collection tube in accordance with another aspect of the present disclosure;

FIG. 6 is a plan view of a blood draw system with a proximal blood diversion chamber coupled to a vascular access device, luer lock access device, and blood culture collection tube in accordance with another aspect of the present disclosure;

FIG. 7 is a plan view of a blood draw system with a proximal blood diversion chamber coupled to a luer lock access device and blood culture collection tube in accordance with another aspect of the present disclosure; and

FIG. 8 is a plan view of a blood draw system with a proximal vented flashback and blood diversion chamber coupled to a luer lock access device and blood culture collection tube

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward an access connector portion of the fluid transfer device, and the terms “in the proximal direction” and “proximally” mean in the direction opposite the direction of the connector.

While not shown or described herein, it is to be understood that the blood sample collection systems described below may be utilized for blood draw from any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood culture sample collection systems for use with PIVCs. However, embodiments of the present disclosure equally extend to use with other catheter devices.

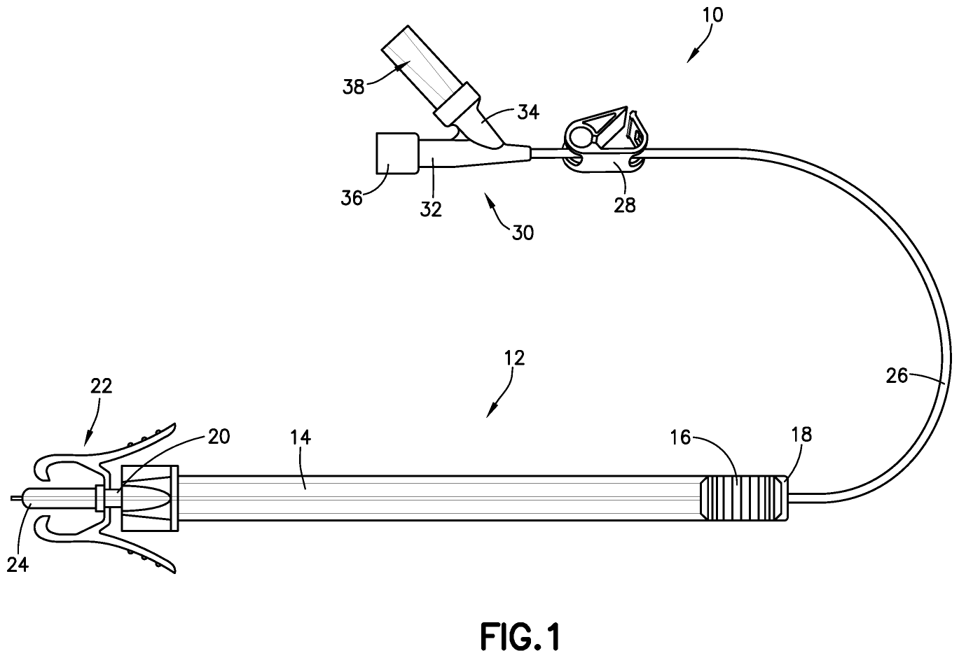

Referring to FIG. 1, a blood draw system 10 in accordance with an aspect of the present disclosure is shown. The blood draw system 10 includes a “line draw” blood draw device 12, which is configured to couple to a PIVC and includes a flexible probe (not shown) that is advanced through the PIVC, beyond the catheter tip, and into a vessel to collect a blood sample. After blood collection, the blood draw device 12 may be removed from the PIVC and discarded. One example of such a blood draw device is known as PIVO™ from Becton, Dickinson and Company. However, it is to be understood that the blood draw system 10 is not limited to such a device, and any “line draw” blood draw device capable of drawing blood or allowing blood to flow therein via an indwelling catheter may be utilized.

The blood draw device 12 includes an introducer body 14 and an actuator 16. The actuator 16 is configured to be linearly displaceable by a clinician along a track or other feature of the introducer body 14 between a proximal end portion 18 and a distal end portion 20 of the introducer body 14. The actuator 16 is operably coupled to an elongated, flexible probe or tube (not shown) to advance and retract the tube through a distal core portion 24 located near the distal end portion 20 of the introducer body 14. That is, displacement of the actuator 16 in a first (distal) direction advances the tube through the distal core portion 24 such that the tube can enter the patient's vasculature when blood draw device 12 is coupled to a vascular access device. Conversely, displacement of the actuator 16 in a second (proximal) direction retracts the tube through the distal core portion 24, ultimately retracting the tube from the patient's vasculature.

The blood draw device also includes a connector member 22 configured for selectively coupling the blood draw device 12 to, e.g., a needleless access connector of an adapter coupled to the vascular access device. In some embodiments, connector member 22 is configured as an alligator clip-type connector, with opposing distal clip portions allowing for securement of the blood draw device 12 to a surface of the needleless access connector. A pair of proximal clip portions are sized and configured to be pinched or otherwise manipulated by a clinician to release the distal clip portions from engagement with the needleless access connector.

Referring still to FIG. 1, a proximal extension tube 26 extends from the proximal end portion 18 of the introducer body 14, with the proximal extension tube 26 being fluidly coupled to the blood draw tube (not shown) of the blood draw device 12. An occlusion device 28 such as, e.g., a clamp may be provided in-line with the proximal extension tube 26 to enable a user to selectively block fluid flow through the proximal extension tube 26.

Additionally, blood draw system 10 includes proximal dual port adapter 30 coupled to a proximal end of the proximal extension tube 26. The proximal dual port adapter 30 includes a first port 32 and a second port 34. In some embodiments, the proximal dual port adapter 30 is configured as a Y-adapter. However, it is to be understood that proximal dual port adapter 30 may be configured in any appropriate manner such as, e.g., a T-adapter, etc. In some embodiments, a removable end cap 36 may be provided over a proximal end portion of the first port 32, with the end cap 36 capable of being selectively removable to expose any appropriate fitting, valve, or connector such as, e.g., a luer connector such that the blood draw system 10 may be coupled to an appropriate blood collection interface such as, e.g., a luer lock access device. However, in other embodiments, the end cap 36 may be omitted.

Blood draw system 10 may further include a vented flashback and diversion chamber 38 coupled to the second port 34 of the proximal dual port adapter 30. As will be described in further detail below, the vented flashback and diversion chamber 38 is configured to divert and store an initial volume of blood that is drawn into the system via the blood draw device 12, as the initial volume of blood may be contaminated by, e.g., bacteria and/or fungi introduced during catheter insertion into the patient's skin and dermal layers or by microbes present in the indwelling IV catheter. While not shown, vented flashback and diversion chamber 38 may include a vent plug or other device at a proximal end thereof to selectively allow air to pass through the vented flashback and diversion chamber 38.

The vented flashback and diversion chamber 38 may have any shape or form with sufficient volume to divert and hold the initial blood volume. In some embodiments, the internal volume of the combined vented flashback and diversion chamber 38 and internal volume of the second port 34 is at least 0.15 mL. This volume is considered sufficient to capture the initial blood sample drawn into the system which may be contaminated. In some embodiments, the combined internal volume of the vented flashback and diversion chamber 38 and the internal volume of the second port 34 is between 0.15 mL and 2.0 mL. In other embodiments, the combined internal volume of the vented flashback and diversion chamber 38 and the internal volume of the second port 34 is between 0.15 mL and 5.0 mL. However, it is to be understood that the internal volume of the vented flashback and diversion chamber 38 is not limited to these examples.

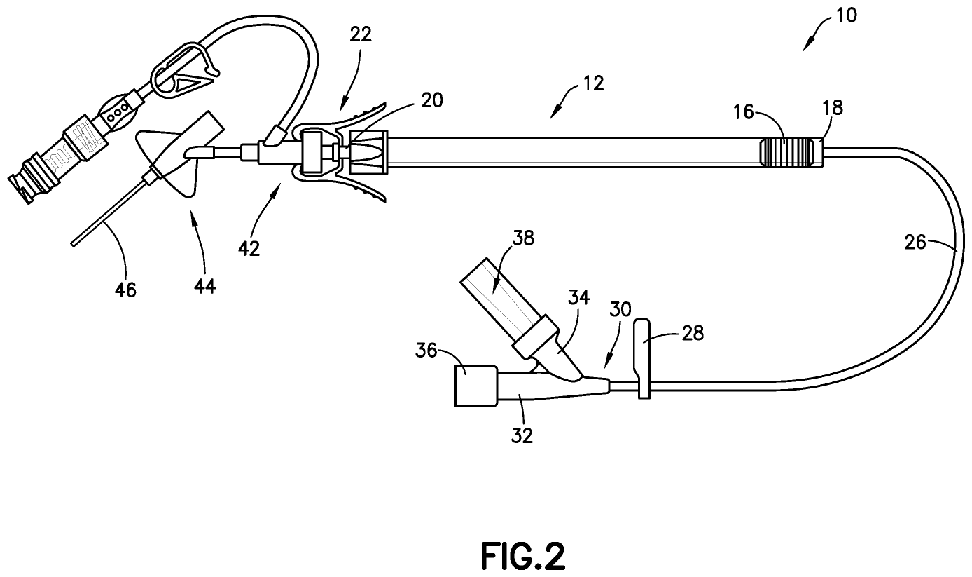

Referring now to FIG. 2, the blood draw system 10 coupled to a vascular access device in a first configuration is illustrated. Specifically, the blood draw device 12 may be coupled to, e.g., a near patient access port 42 via a needle-free connection. The near patient access port 42 is in fluid communication with a catheter adapter 44 having a catheter 46 extending distally therefrom. As noted above, the blood draw system 10 may be used with any appropriate vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

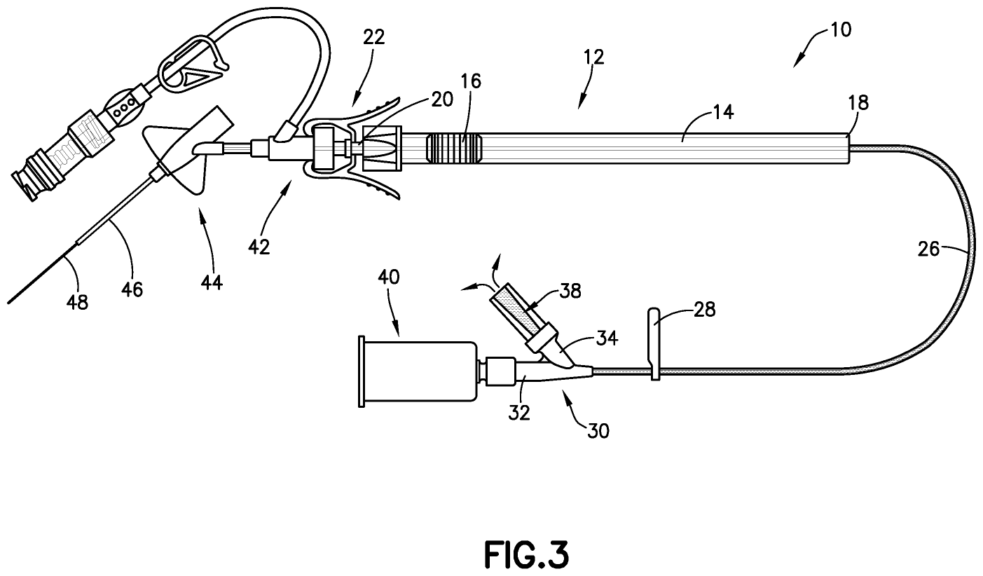

FIG. 3 illustrates the blood draw system 10 in a second configuration. With the blood draw device 12 coupled to the near patient access port 42, a blood collection interface such as, e.g., a luer lock access device (LLAD) 40 may be coupled to the first port 32 of the proximal dual port adapter 30. With the luer lock access device 40 in place, the clinician may advance a blood draw tube 48 housed within the blood draw device 12 through both the catheter adapter 44 and the catheter 46 by distal advancement of the actuator 16 along the introducer body 14. At its fully advanced position, the blood draw tube 48 extends to or beyond a distal tip of the indwelling catheter 46 and into a high blood flow location in the patient's vein, thereby providing a fluid path for venous blood to be drawn via the blood draw device 12.

With the blood draw tube 48 in this advanced position shown in FIG. 3, vented flashback and diversion chamber 38 extending from the second port 34 is vented, either automatically or manually. This venting of the vented flashback and diversion chamber 38 enables an initial volume of blood to flow through the tube 48, to the proximal extension tube 26, and into the vented flashback and diversion chamber 38, with blood flow stopping at a vent portion (not shown) of the vented flashback and diversion chamber 38. In this way, the system 10 is primed with the initial volume of blood isolated within the vented flashback and diversion chamber 38, and the system is in condition for connection to, e.g., a blood culture vacuum tube for sample collection through the blood sample fluid path and the luer lock access device 40. However, because a collection container is not yet coupled to the luer lock access device 40, the flow of blood stops at the vented flashback and diversion chamber 38.

In some embodiments, air venting and blood flow into the vented flashback and diversion chamber 38 stops when blood contacts the venting portion (not shown). The venting portion may be formed of, e.g., a membrane, paper, porous, film, or mechanical features that allows air to pass therethrough, but stops fluid from passing therethrough when wetted. When venting portion is wetted, air is prevented from being pulled into the vented flashback and diversion chamber 38, which prevents the volume of blood held within the vented flashback and diversion chamber 38 from entering the first port 32, thus preventing the potentially contaminated initial volume of blood from entering a blood collection container coupled to the luer lock access device 40. However, alternative means of isolating the initial blood sample within the vented flashback and diversion chamber 38 are also possible in accordance with other embodiments of the present disclosure. For example, in some embodiments, the initial blood sample may be manually isolated via a mechanical occlusion (i.e., via a clamp in a distal portion of the vented flashback and diversion chamber 38). In some embodiments, a one-way venting/fluid or check valve may be provided at the entrance of the vented flashback and diversion chamber 38, thereby allowing an initial blood sample to flow therein, but preventing outflow of the blood sample therefrom.

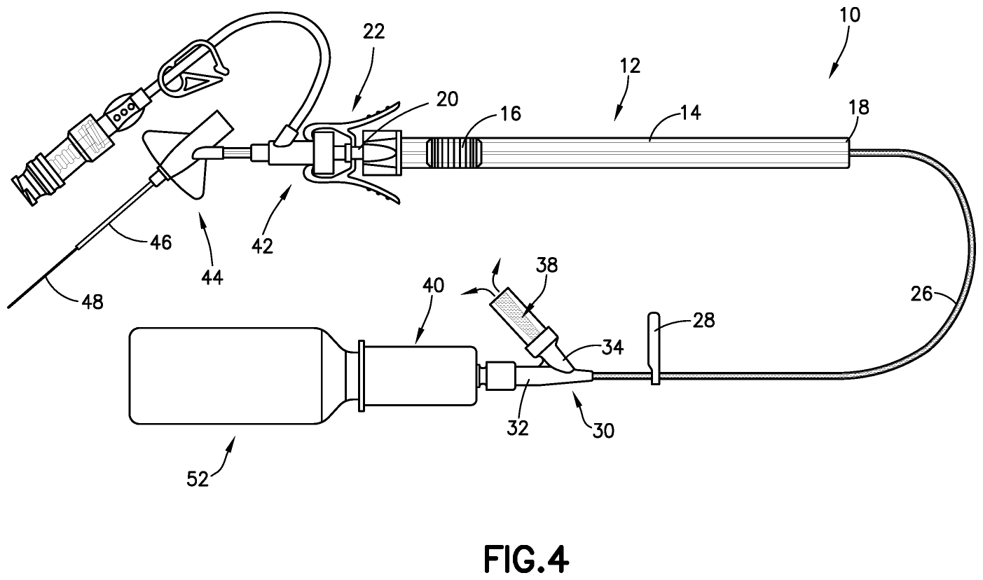

Next, referring to FIG. 4, the blood draw system 10 coupled to a vascular access device in a third configuration is illustrated. In the third configuration shown in FIG. 4, a blood collection container 52 is fluidly coupled to the luer lock access device 40, thereby allowing a blood sample from the patient's vasculature to flow through the extension tube 26 via the first port 32 of the proximal dual port adapter 30. The blood collection container 52 may be any appropriate container such as, e.g., a BD BACTEC™ blood culture collection container, an evacuated tube, a syringe, etc. As noted above, the initial volume of blood is isolated within the vented flashback and diversion chamber 38, thereby preventing this potentially contaminated initial volume of blood from passing through the first port and to the blood collection container 52, aiding in the prevention of false positive blood culture tests.

Once the desired blood sample is collected into the blood collection container 52, the blood collection container 52 may be disconnected from the luer lock access device 40 and sent for analysis. If additional blood samples are needed, one or more blood collection containers 52 may be coupled to the luer lock access device 40 to draw the desired samples. With each of these sample collections, the initial (and potentially contaminated) volume of blood remains within the vented flashback and diversion chamber 38.

The blood draw system 10 described above with respect to FIGS. 1-4 is configured with a luer lock access device 40 and extension tube 26 to allow for greater flexibility during blood collection and to enable upright positioning of the blood collection container 52 during sample collection. In some embodiments, the blood draw system 10 can include a luer lock access device 40 having integrated extension tubing. In other embodiments, the luer lock access device 40 may have removably attached extension tubing. In some embodiments, the fluid path of all or some of the extension tube 26 may be optimized to reduce hemolysis during blood culture sample collection and/or during subsequent vacuum tube or syringe-based blood sample collections after the blood culture sample collection.

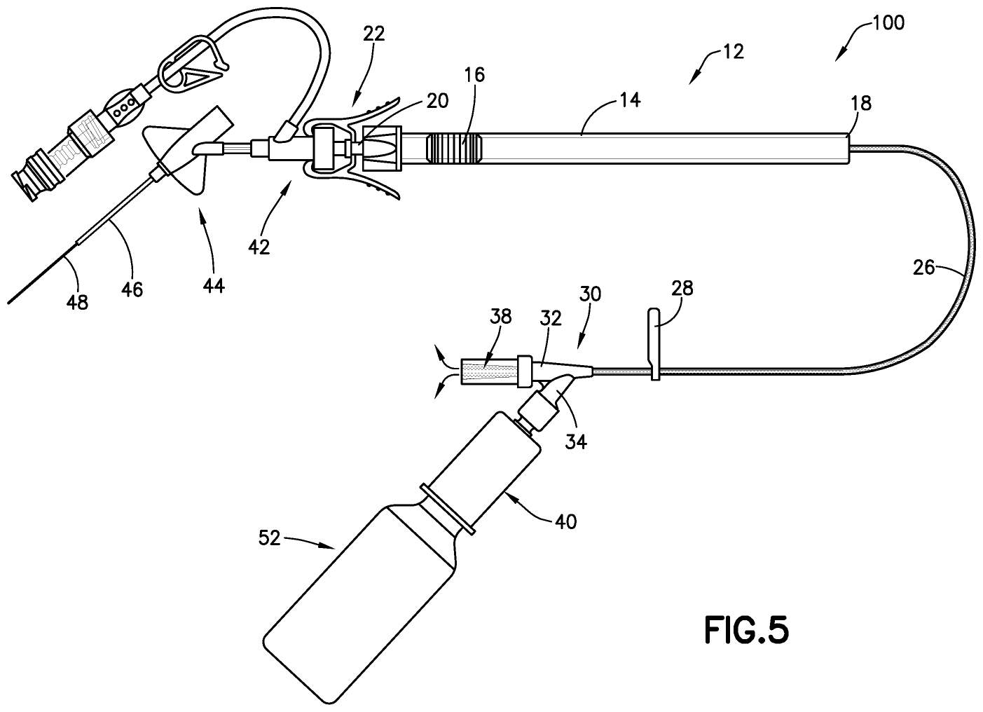

Next, referring to FIG. 5, a blood draw system 100 in accordance with another aspect of the present disclosure is shown. Blood draw system 100 includes the same features as blood draw system 10 described above with respect to FIGS. 1-4. However, in blood draw system 100, the vented flashback and diversion chamber 38 is coupled to the first port 32 of the proximal dual port adapter 30, while the luer lock access device 40 is coupled to the second port 34, which extends at an angle from the first port 32. Thus, it is to be understood that the vented flashback and diversion chamber 38 is capable of collecting and retaining an initial blood sample prior to connection of a blood collection container 52, regardless of whether the vented flashback and diversion chamber 38 is coupled to the first port 32 positioned in-line with the extension tube 26 (as shown in FIG. 5) or the second port 34 (as shown in FIGS. 1-4).

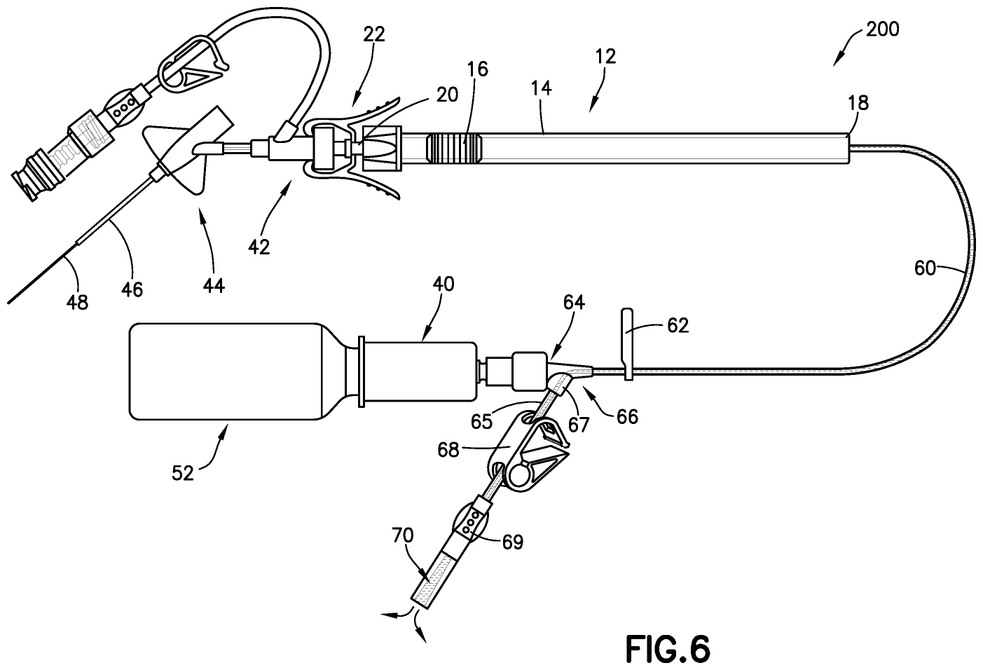

Referring to FIG. 6, a blood draw system 200 in accordance with another aspect of the present disclosure is shown. Similar to blood draw system 10 and blood draw system 100 described above, blood draw system 200 is configured for use with a blood draw device 12 coupled to a near patient access port 42 in fluid communication with a catheter adapter 44. However, rather than a proximal dual port adapter, blood draw system 200 includes a y-site connector 66 configured to be coupled to a proximal end of proximal extension tube 60. A coupler interface 64 is provided on the y-site connector 66 for selective attachment of, e.g., a luer lock access device 40. Additionally, the y-site connector 66 includes a side port 67, with a secondary extension tube 65 extending from the side port 67. A proximal end of the secondary extension tube 65 may include a connector 69, with the connector 69 configured to receive a vented flashback and diversion chamber 70.

As shown in FIG. 6, a first occlusion device 62 may be provided in-line with the proximal extension tube 60 to enable selective occlusion of the fluid flow through proximal extension tube 60, while a second occlusion device 68 may be provided in line with the secondary extension tube 65 to enable selective occlusion of the fluid flow through secondary extension tube 65.

Prior to the blood collection container 52 being coupled to the luer lock access device 40, the blood draw tube 48 is extended to the advanced position beyond catheter 46, as shown in FIG. 6. The flashback and diversion chamber 70 is then vented, either automatically or manually. This venting of the vented flashback and diversion chamber 70 enables an initial volume of blood to flow through the tube 48, to the proximal extension tube 60, though the secondary extension tube 65, and into the vented flashback and diversion chamber 70, with blood flow stopping at a vent portion (not shown) of the vented flashback and diversion chamber 70. In this way, an initial volume of blood isolated within the vented flashback and diversion chamber 70 and the secondary extension tube 65. Then, when the blood collection container 52 is coupled to the luer lock access device 40, blood flows through the proximal extension tube 60 to the blood collection container 52, with the initial volume of blood remaining isolated within the vented flashback and diversion chamber 70 and the secondary extension tube 65 such that potentially contaminated blood does not enter the blood collection container 52. As noted above, the second occlusion device 68 may be clamped or otherwise closed so as to ensure that the initial volume of blood remains isolated from the blood sample collected in the blood collection container 52.

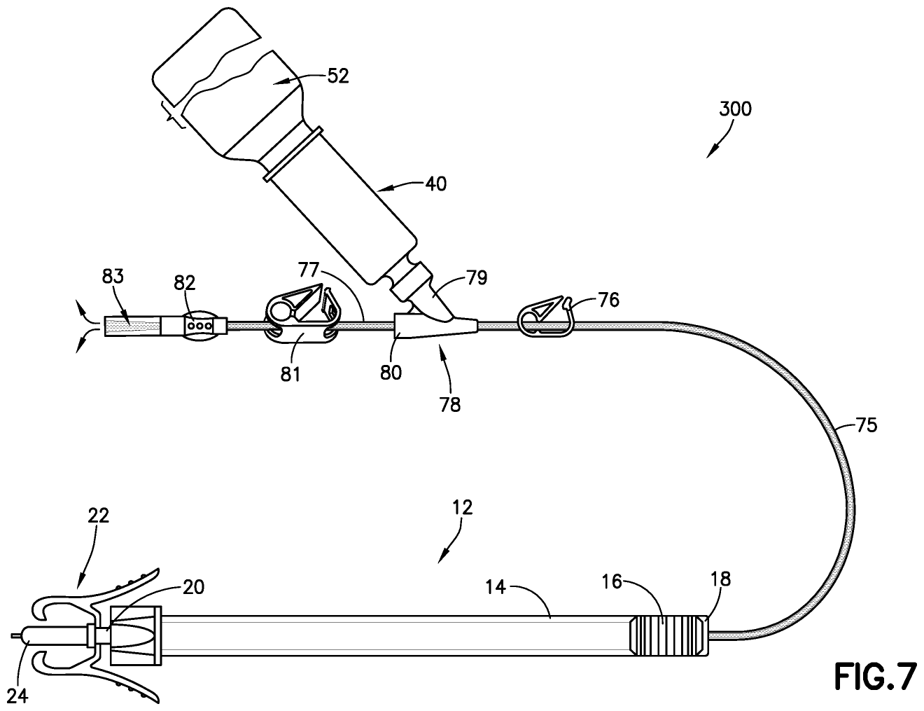

Next, referring to FIG. 7, a blood draw system 300 in accordance with another aspect of the present disclosure is shown. Blood draw system 300 is configured for use with a blood draw device 12 as described above with respect to FIG. 1, with blood draw device 12 capable of being coupled to a near patient access port (not shown) in fluid communication with a catheter adapter (not shown). Blood draw system 300 includes a proximal dual port adapter 78 configured to be coupled to a proximal end of proximal extension tube 75. The dual port adapter 78 includes a first port 80 and a second port 79, with the first port 80 extending substantially in-line with the proximal end portion of the proximal extension tube 75, while the second port 79 extends at an angle relative to the first port 80 so as to form, e.g., a Y-shaped adapter. A luer lock access device 40 may be coupled to the second port 79, with the luer lock access device configured to receive, e.g., a blood collection container 52. Additionally, a secondary extension tube 77 may extend from the first port 80. A proximal end of the secondary extension tube 77 may include a connector 82, with the connector 82 configured to receive a vented flashback and diversion chamber 83.

As shown in FIG. 7, a first occlusion device 76 may be provided in-line with the proximal extension tube 75 to enable selective occlusion of the fluid flow through proximal extension tube 75, while a second occlusion device 81 may be provided in line with the secondary extension tube 77 to enable selective occlusion of the fluid flow through secondary extension tube 77.

Prior to the blood collection container 52 being coupled to the luer lock access device 40, a blood draw tube (not shown) of the blood draw device 12 is extended to an advanced position beyond the distal end of an indwelling catheter (not shown). The flashback and diversion chamber 83 is then vented, either automatically or manually. This venting of the vented flashback and diversion chamber 83 enables an initial volume of blood to flow through the tube extending into the patient's vasculature, to the proximal extension tube 75, though the secondary extension tube 77, and into the vented flashback and diversion chamber 83, with blood flow stopping at a vent portion (not shown) of the vented flashback and diversion chamber 83. In this way, an initial volume of blood isolated within the vented flashback and diversion chamber 83 and the secondary extension tube 77. Then, when the blood collection container 52 is coupled to the luer lock access device 40, blood flows through the proximal extension tube 75 to the blood collection container 52, with the initial volume of blood remaining isolated within the vented flashback and diversion chamber 83 and the secondary extension tube 77 such that potentially contaminated blood does not enter the blood collection container 52. As noted above, the second occlusion device 82 may be clamped or otherwise closed so as to ensure that the initial volume of blood remains isolated from the blood sample collected in the blood collection container 52.

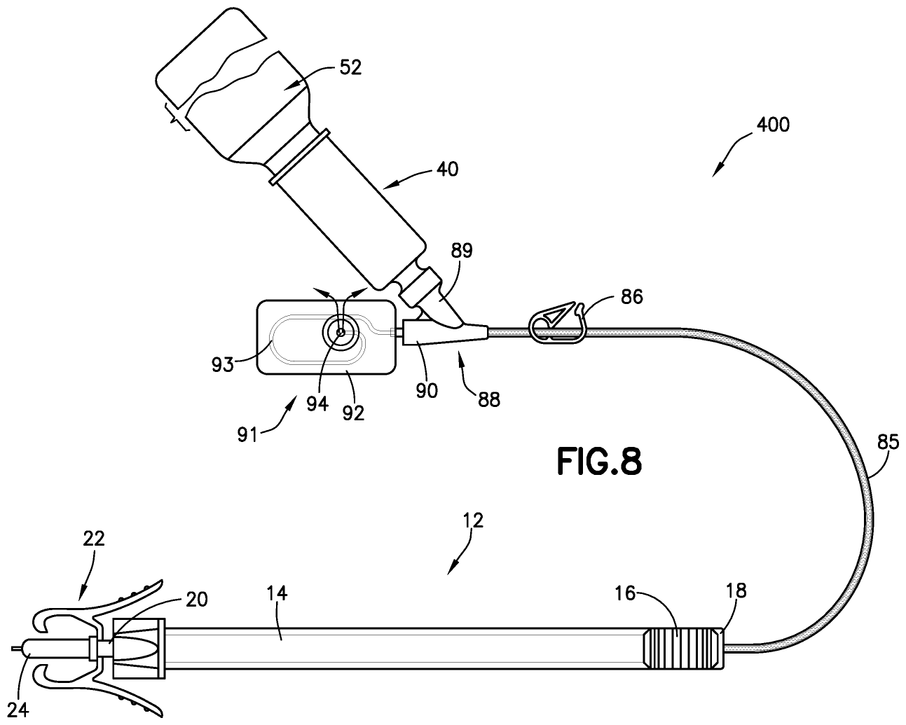

Referring now to FIG. 8, a blood draw system 400 in accordance with another aspect of the present disclosure is shown. Blood draw system 400 is configured for use with a blood draw device 12 as described above with respect to FIG. 1, with blood draw device 12 capable of being coupled to a near patient access port (not shown) in fluid communication with a catheter adapter (not shown). Blood draw system 400 includes a proximal dual port adapter 88 configured to be coupled to a proximal end of proximal extension tube 85. The dual port adapter 88 includes a first port 90 and a second port 89, with the first port 90 extending substantially in-line with the proximal end portion of the proximal extension tube 85, while the second port 89 extends at an angle relative to the first port 90 so as to form, e.g., a Y-shaped adapter. A luer lock access device 40 may be coupled to the second port 89, with the luer lock access device 40 configured to receive, e.g., a blood collection container 52.

Blood draw system 400 further includes a vented flashback and diversion device 91 coupled to the first port 90 of the proximal dual port adapter 88. In the embodiment shown in FIG. 8, the vented flashback and diversion device 91 includes a body 92, a vented diversion and isolation chamber 93, and a venting portion 94. In one embodiment, the venting portion 94 is configured to automatically vent air when the blood draw device 12 is coupled to a vascular access device and the blood draw tube of the blood draw device 12 is advanced into the patient's vasculature, thereby allowing an initial volume of blood to flow through the proximal extension tube 85 and to the vented diversion and isolation chamber 93. Alternatively, in other embodiments, the venting portion 94 may be manually vented when the blood draw device 12 is coupled to a vascular access device.

The vented diversion and isolation chamber 93 may have any shape or form with sufficient volume to divert and hold the initial blood volume. In some embodiments, the internal volume of the vented diversion and isolation chamber 93 is at least 0.15 mL. This volume is considered sufficient to capture the initial blood sample drawn into the system which may be contaminated. In some embodiments, the internal volume of the vented diversion and isolation chamber 93 is between 0.15 mL and 2.0 mL. In other embodiments, the internal volume of the vented diversion and isolation chamber 93 is between 0.15 mL and 5.0 mL. However, it is to be understood that the internal volume of the vented diversion and isolation chamber 93 is not limited to these examples.

As shown in FIG. 8, an occlusion device 86 may be provided in-line with the proximal extension tube 85 to enable selective occlusion of the fluid flow through proximal extension tube 85.

Prior to the blood collection container 52 being coupled to the luer lock access device 40, a blood draw tube (not shown) of the blood draw device 12 is extended to an advanced position beyond the distal end of an indwelling catheter (not shown). The vented diversion and isolation chamber 93 is then vented, either automatically or manually. This venting of the vented diversion and isolation chamber 93 enables an initial volume of blood to flow through the tube extending into the patient's vasculature, to the proximal extension tube 85, and into the vented diversion and isolation chamber 93, with blood flow stopping at a venting portion 94 of the vented flashback and diversion device 91. In this way, an initial volume of blood isolated within the vented diversion and isolation chamber 93. Then, when the blood collection container 52 is coupled to the luer lock access device 40, blood flows through the proximal extension tube 85 to the blood collection container 52, with the initial volume of blood remaining isolated within the vented diversion and isolation chamber 93 such that potentially contaminated blood does not enter the blood collection container 52.

Using the blood draw systems 10, 100, 200, 300, and 400 described above with respect to FIGS. 1-8, numerous advantages over conventional methods of blood culture sample collection may be realized. First, the number of needle insertions for the patient may be reduced, thereby improving patient comfort and experience, as an existing vascular access device is used for blood culture sample collection. Additionally, using a blood draw device such as, e.g., PIVO™ from Becton, Dickinson and Company, enables the blood draw tube to extend beyond the distal tip of the catheter, therefore reducing the risk of false positives from microbes that may be present in the catheter fluid path of an indwelling catheter.

Additionally, the blood draw systems have built-in automatic and passive diversion and capture of initial blood flow, which may be contaminated due to the presence of bacteria and/or fungi on the patient's skin, hair follicles, and dermal layers. This initial blood flow may be passively or manually isolated within a flashback and diversion chamber. Accordingly, the blood draw system eliminates the need for a separate blood discard sample to be collected, thereby reducing collection steps and improving workflow, while also reducing the opportunity for contamination during draw due to a reduced number of connections made with the blood draw system. Also, while existing blood culture collection devices with diversion chambers may only be used with a catheter immediately at the time of insertion, the blood draw systems described above may extend the use of blood collection devices with diversion chambers to any period throughout the catheter's dwell time.

The blood draw systems 10, 100, 200, 300, and 400 are also compatible with standard luer lock access devices, thereby enabling vacuum tube blood collection immediately after blood culture sample collection. Furthermore, the blood draw systems 10, 100, 200, 300, and 400 may be provided with optimized fluid paths for reduced hemolysis of the blood samples in subsequent blood collection samples after the initial blood culture sample collection.

While several embodiments of blood draw systems configured for blood sample collection from an indwelling catheter were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A blood draw system, comprising:
a blood draw device having a distal end portion and a proximal end portion, wherein the blood draw device comprises an actuator and a blood draw tube operably coupled to the actuator, wherein the actuator is configured to selectively advance the blood draw tube through a catheter of a vascular access device when the blood draw device is coupled to the vascular access device;
an extension tube extending from the proximal end portion of the blood draw device, wherein the extension tube is in fluid communication with the blood draw tube of the blood draw device;
a dual port adapter coupled to a proximal end of the extension tube, wherein the dual port adapter comprises a first port and a second port; and
a vented flashback and diversion chamber coupled to one of the first port and the second port, wherein the vented flashback and diversion chamber is configured to receive and hold an initial volume of blood drawn through the extension tube by the blood draw device.

2. The system of claim 1, wherein the vented flashback and diversion chamber further comprises a venting portion.

3. The system of claim 2, wherein the venting portion is configured to vent air when the blood draw device is coupled to the vascular access device and the blood draw tube of the blood draw device is advanced into a patient's vasculature to draw the initial volume of blood into the vented flashback and diversion chamber.

4. The system of claim 3, wherein the venting portion is configured to vent air automatically when the blood draw device is coupled to the vascular access device and the blood draw tube of the blood draw device is advanced into a patient's vasculature.

5. The system of claim 3, wherein the venting portion is manually vented when the blood draw device is coupled to the vascular access device and the blood draw tube of the blood draw device is advanced into a patient's vasculature.

6. The system of claim 3, wherein the venting portion is formed of at least one of a membrane, paper, porous material, film, or mechanical feature that allows air to pass therethrough but prevents fluid to pass therethrough when wetted.

7. The system of claim 1, further comprising a removable end cap positionable on the other one of the first port and the second port.

8. The system of claim 1, further comprising a luer lock access device coupled to the other one of the first port and the second port.

9. The system of claim 1, further comprising a secondary extension tube, wherein a distal end of the secondary extension tube is coupled to one of the first port and the second port and a proximal end of the secondary extension tube is coupled to a connector.

10. The system of claim 9, wherein the vented flashback and diversion chamber is coupled to the connector.

11. The system of claim 9, further comprising a fluid occlusion device positioned in-line with the secondary extension tube, wherein the fluid occlusion device is configured to selectively occlude the flow of blood from the vented flashback and diversion chamber.

12. The system of claim 1, wherein the blood draw device further includes an introducer body, and wherein the actuator is configured to move linearly along the introducer body to advance and retract the blood draw tube from the distal end portion of the blood draw device.

13. The system of claim 1, wherein the vented flashback and diversion chamber and the second port have a combined internal volume of at least 0.15 mL.

14. A method of using a blood sample collection system, comprising:
providing the blood sample collection system, the system comprising:
a blood draw device having a distal end portion and a proximal end portion, wherein the blood draw device comprises an actuator and a blood draw tube operably coupled to the actuator,
an extension tube extending from the proximal end portion of the blood draw device, wherein the proximal extension tube is in fluid communication with the blood draw tube of the blood draw device;
a dual port adapter coupled to a proximal end of the extension tube, wherein the dual port adapter comprises a first port and a second port; and
a vented flashback and diversion chamber coupled to one of the first port and the second port;
coupling the blood draw device to a vascular access device having an indwelling catheter;
advancing the blood draw tube of the blood draw device through the vascular access device and beyond a distal tip of the indwelling catheter; and
allowing the initial volume of blood to flow through the blood draw tube and the extension tube and into the vented flashback and diversion chamber of the blood sample collection system.

15. The method of claim 14, further comprising venting the vented flashback and diversion chamber prior to allowing the initial volume of blood to flow therein.

16. The method of claim 15, wherein venting the vented flashback and diversion chamber comprises manual venting.

17. The method of claim 15, wherein venting the vented flashback and diversion chamber comprises automatic venting.

18. The method of claim 14, further comprising:
coupling a blood collection interface to the other one of the first port and the second port of the dual port adapter.

19. The method of claim 18, further comprising:
coupling a first blood collection container to the blood collection interface after the initial volume of blood is collected in the vented flashback and diversion chamber, and
collecting a first blood sample within the first blood collection container.

20. The method of claim 19, further comprising:
removing the first blood collection container from the blood collection interface,
coupling a second blood collection container to the blood collection interface, and
collecting a second blood sample within the second blood collection container.
